(19) United States
(12) Patent Application Publication
(10) Pub. No.: US 2009/0275761 A1
(43) Pub. Date: Nov. 5, 2009

(54) Catalytic Preparation of Cyclic Carboxylic Esters

(76) Inventor: Felix Spindler, Starrkirch-Wil (CH)

Correspondence Address: WENDEROTH, LIND & PONACK, L.L.P., 1030 15th Street, N.W.,, Suite 400 East, Washington, DC, 20005-1503, US

(21) Appl. No.: 11/887,447

(22) PCT Filed: Apr. 7, 2006
(86) PCT No.: PCT/EP2006/061424

§ 371 Date: Sep. 28, 2007

(30) Foreign Application Priority Data
Apr. 8, 2005 (CH) 00641/05

(51) Int. Cl. C07D 307/93

(52) U.S. Cl. 549/302

(57) ABSTRACT

Preparation of cyclic esters by hydrogenation of a carbonyl group in at least one anhydride radical —C(O)—O—C(O)— of a cyclic dicarboxylic or polycarboxylic anhydride by means of hydrogen in the presence of a homogeneous noble metal catalyst, characterized in that the hydrogenation is carried out in a homogeneous reaction mixture using an iridium catalyst. The cyclic esters are obtained in good chemical and optical yields when prochiral anhydrides are used together with chiral iridium catalysts.

The present invention relates to a process for preparing cyclic monocarboxylic esters by hydrogenation of cyclic dicarboxylic anhydrides by means of hydrogen in the presence of a homogeneous noble metal catalyst, with the noble metal being iridium.

In Bull. Chem. Soc. Jpn. 72 (1999), pages 573 to 580, K. Nagayama et al. describe the cleavage of benzoic anhydride into benzaldehyde and benzoic acid in the hydrogenation by means of hydrogen in the presence of Pd[P(C₆H₅)₃]₄ as homogeneous catalyst.

In J. Organometal. Chem. 188 (1980), pages 109 to 119, M. Bianchini et al. describe the quantitative hydrogenation of succinic anhydride to γ-valerolactone by means of hydrogen in the presence of [Ru₄H₄(CO)₈[P(n-butyl)₃]₄] as homogeneous catalyst.

In Bull. Chem. Soc. Jpn. 57 (1984), pages 897 to 898, T. Ikariya et al. describe the hydrogenation of 2,2-dimethylglutaric anhydride to 2,2-dimethyl-δ-caprolactone by means of hydrogen in the presence of a homogeneous ruthenium catalyst. The yield is low, but the chemoselectivity is excellent.

In Tetrahedron Letters, 22(43), pages 4297 to 4300 (1981) K. Osakada et al. describe the hydrogenation of prochiral cycloalkane-1,2-dicarboxylic anhydrides or prochiral glutaric anhydrides substituted in the 3 position to γ-valerolactones or δ-caprolactones substituted in the 3 position by means of hydrogen in the presence of triethylamine and a homogeneous ruthenium catalyst containing chiral diphosphine ligands (here DIOP). The yields and the chemoselectivities are moderate, and the optical yields achieved are reported as only up to 20% ee.

The processes described hitherto are therefore not suitable for industrial production processes. Pharmaceutical active compounds and agrochemicals (pesticides) and natural materials for this purpose frequently contain lactone rings and sometimes asymmetric carbon atoms. There is a need for the catalytic preparation of such active compounds or intermediates for such active compounds in good chemical and, if appropriate, optical yields.

It has surprisingly now been found that excellent chemoselectivites and outstanding enantioselectivities and also high chemical and optical yields can be achieved in the hydrogenation of possibly prochiral cyclic dicarboxylic anhydrides by means of hydrogen to form lactones when the hydrogenation is carried out in the presence of homogeneous iridium catalysts using, if appropriate, chiral ligands. Furthermore, it has been found that the reaction can, under suitable reaction conditions, be stopped after formation of an intermediate (cyclic hydroxy ester) and the intermediate formed can then be reacted further to give the desired cyclic ester.

The present invention provides a process for preparing cyclic esters, with the exception of cyclic esters of the formula

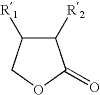

where R′₁ and R′₂ are each, independently of one another, an —NR′₃R′₄ group and R′₃ and R′₄ are each, independently of one another, hydrogen, unsubstituted or substituted alkyl, unsubstituted or substituted alkenyl, unsubstituted or substituted cycloalkyl, unsubstituted or substituted aryl, unsubstituted or aromatic-substituted aralkyl, unsubstituted or aromatic-substituted aralkenyl, cycloalkylalkyl which is unsubstituted or substituted in the cycloalkyl, heterocyclyl, unsubstituted or substituted alkanoyl, unsubstituted or substituted aroyl, unsubstituted or substituted alkylsulphonyl, unsubstituted or substituted arylsulphonyl or a silyl group Si(alkyl)₃, Si(aryl)₃ or Si(alkyl)_{1 or 2}(aryl)_{2 or 1}, or the two radicals R′₃ together are —C(O) and the two radicals R′₄ are each, independently of one another, as defined above, by hydrogenation of a carbonyl group in at least one anhydride radical —C(O)—O—C(O)— of a cyclic dicarboxylic or polycarboxylic anhydride by means of hydrogen in the presence of a homogeneous noble metal catalyst, which is characterized in that the hydrogenation is carried out in a homogeneous reaction mixture using an iridium catalyst.

The process of the invention can be carried out at low or elevated temperatures, for example temperatures of from −20 to 150° C., preferably from −10 to 100° C. and particularly preferably from 10 to 80° C. The optical yields are generally better at relatively low temperature than at higher temperatures, while a faster conversion can be achieved at higher temperatures.

The process of the invention can be carried out at atmospheric pressure or superatmospheric pressure. The pressure can be, for example, from 10⁵ to 2×10⁷ Pa (pascal).

Catalysts are preferably used in amounts of from 0.0001 to 10 mol %, particularly preferably from 0.001 to 10 mol % and very particularly preferably from 0.01 to 5 mol %, based on the cyclic anhydride.

The hydrogenation can be carried out without solvents or in the presence of an inert solvent, with it being possible to use one solvent or mixtures of solvents. Suitable solvents are, for example, aliphatic, cycloaliphatic and aromatic hydrocarbons (pentane, hexane, petroleum ether, cyclohexane, methylcyclohexane, benzene, toluene, xylene), ethers (diethyl ether, dibutyl ether, t-butyl methyl ether, ethylene glycol dimethyl ether, ethylene glycol diethyl ether, diethylene glycol dimethyl ether, tetrahydrofuran, dioxane, diethylene glycol monomethyl or monoethyl ether), ketones (acetone, methyl isobutyl ketone), carboxylic esters and lactones (ethyl or methyl acetate, valerolactone), N-substituted lactams (N-methylpyrrolidone), carboxamides (dimethylamide, dimethylformamide), acyclic ureas (dimethylimidazoline) and sulphoxides and sulphones (dimethyl sulphoxide, dimethyl sulphone, tetramethylene sulphoxide, tetramethylene sulphone) and unfluorinated or fluorinated alcohols (methanol, ethanol, propanol, butanol, ethylene glycol monomethyl ether, ethylene glycol monoethyl ether, diethylene glycol monomethyl ether, 1,1,1-trifluoroethanol) and water. Further suitable solvents are low molecular weight carboxylic acids such as acetic acid. The solvents can be used alone or in mixtures of at least two solvents. Preferred solvents are hydrocarbons, alcohols, ethers and mixtures of at least 2 such solvents.

The reaction can be carried out in the presence of cocatalysts, for example alkali metal halides (Li, K, Na) or ammonium halides, in particular quaternary ammonium halides, with halide preferably being Br or I and particularly preferably being I. Tetrabutylammonium iodide has been found to be particularly useful. The amount of cocatalysts can be, for example, from 0.1 to 100 equivalents and preferably from 10 to 80 equivalents, based on the iridium complex. The hydrogenation can also be carried out in the presence of cocatalysts and protic acids, for example mineral acids, carboxylic acids or sulphonic acids (for cocatalysts and acids, see, for example, U.S. Pat. No. 5,371,256, U.S. Pat. No. 5,446,844, U.S. Pat. No. 5,583,241 and EP-A-0 691 949). The acid can, for example, be used as solvent or in amounts of from 0.001 to 50% by weight and preferably from 0.1 to 50% by weight, based on the amount of imine.

The presence of fluorinated alcohols such as 1,1,1-trifluoroethanol can likewise promote the catalytic reaction.

The metal complexes used as catalysts can be added as separately prepared, isolated compounds or they can be formed in situ prior to the reaction and then mixed with the substrate to be hydrogenated. In the reaction using isolated metal complexes, it can be advantageous for ligands to be additionally added or for an excess of ligands to be used in the in-situ preparation. The excess can be, for example, from 1 to 6 mol and preferably from 1 to 2 mol, based on the iridium compound used for the preparation.

The process of the invention is generally carried out by initially charging the catalyst and then adding the substrate and if appropriate a reaction auxiliary, pressurizing the reaction vessel with hydrogen and then starting the reaction. The process can be carried out continuously or batchwise in various types of reactors.

The cyclic anhydrides can, depending on the structure and size of the skeleton, contain one or more cyclic anhydride groups, for example from one to four, more preferably from one to three and particularly preferably one or two, cyclic anhydride groups. Furthermore, the cyclic anhydride group can have a total of from 4 to 10, preferably from 5 to 8, more preferably from 5 to 7 and particularly preferably 5 or 6, ring atoms. The cyclic anhydrides themselves can have a total of up to 60, preferably up to 40 and particularly preferably up to 30, carbon atoms in the skeleton, with carbon atoms being able to be replaced by heteroatoms and/or groups of heteroatoms, for example —C(═O)—, —O—, —S—, —S(═O)—, —S(O)₂, ═N—, —HN— or —RₐN—, where Rₐ is preferably C₁-C₈-alkyl, C₅-C₈-cycloalkyl, C₅-C₈-cycloalkyl-C₁-C₄-alkyl, C₆-C₁₀-aryl, C₇-C₁₂-aralkyl or C₁-C₈-acyl.

The skeleton to which the at least one anhydride group —C(O)—O—C(O)— is bound can comprise, for example, aliphatic, heteroaliphatic, cycloaliphatic, heterocycloaliphatic, cycloaliphatic-aliphatic, heterocycloaliphatic-aliphatic, aromatic, heteroaromatic, aromatic-aliphatic or heteroaromatic-aliphatic radicals, with the cyclic radicals having, for example, from 3 to 8, preferably from 5 to 7 and particularly preferably 5 or 6, ring atoms. The cyclic radicals mentioned may be bridged, fused or bridged and fused to form polycyclic radicals. Such ring systems can contain, for example, from 2 to 6 and preferably from 2 to 4 cyclic and/or heterocyclic hydrocarbon radicals. Heteratoms in heteroradicals can, for example, be selected from the group consisting of —O—, —S—, ═N—, —HN— and —RₐN—, where Rₐ is preferably C₁-C₈-alkyl, C₅-C₈-cycloalkyl, C₅-C₈-cycloalkyl-C₁-C₄-alkyl, C₆-C₁₀-aryl, C₇-C₁₂-aralkyl or C₁-C₈-acyl.

The skeleton of the cyclic anhydrides can be unsubstituted or substituted by one or more substituents which may in turn be unsubstituted or substituted. The number of substituents is dependent on, inter alia, the size of the molecule. The number of substituents can be, for example, up to 15, preferably up to 10 and particularly preferably from 1 to 5. The substituents are advantageously inert under the reaction conditions.

Substituted or unsubstituted substituents can, for example, be C₁-C₁₂-alkyl, preferably C₁-C₈-alkyl and particularly preferably C₁-C₄-alkyl. Examples are methyl, ethyl, n- or i-propyl, n-, i- or t-butyl, pentyl, hexyl, hepthyl, octyl, decyl and dodecyl.

Substituted or unsubstituted substituents can, for example, be C₅-C₈-cycloalkyl, preferably C₅-C₆-cycloalkyl. Examples are cyclopentyl, cyclohexyl and cyclooctyl.

Substituted or unsubstituted substituents can, for example, be C₅-C₈-cycloalkyl-alkyl, preferably C₅-C₆-cycloalkyl-alkyl. Examples are cyclopentylmethyl, cyclohexylmethyl or cyclohexylethyl and cyclooctylmethyl.

Substituted or unsubstituted substituents can, for example, be C₆-C₁₈-aryl and preferably C₆-C₁₀aryl. Examples are phenyl and naphthyl.

Substituted or unsubstituted substituents can, for example, be C₇-C₁₂-aralkyl (for example benzyl or 1-phenyleth-2-yl).

Substituted or unsubstituted substituents can, for example, be tri(C₁-C₄-alkyl)Si or triphenylsilyl. Examples of trialkylsilyl are trimethylsilyl, triethylsilyl, tri-n-propylsilyl, tri-n-butylsilyl and dimethyl-t-butylsilyl.

Possible substituents are, for example, halogen. Examples are F, Cl and Br.

Substituted or unsubstituted substituents can, for example, be —OH, —SH, —CH(O), —CN, —NCO, —OCN or —NR₀₃R₀₄ where R₀₃, R₀₄ are each, independently of one another, hydrogen, C₁-C₄-alkyl, cyclopentyl, cyclohexyl, phenyl, benzyl or R₀₃ and R₀₄ together form tetramethylene, pentamethylene or 3-oxapentane-1,5-diyl.

A substituted or unsubstituted substituent can, for example, be an oxyl radical, thio radical or sulphoxide or sulphone radical of the formula —OR₀₁, —SR₀₁, —S(O)R₀₁ or —S(O)₂R₀₁ where R₀₁ is hydrogen, C₁-C₁₂-alkyl, preferably C₁-C₈-alkyl and particularly preferably C₁-C₄-alkyl; C₅-C₈-cycloalkyl, preferably C₅-C₆-cycloalkyl; C₆-C₁₈-aryl and preferably C₆-C₁₀-aryl; or C₇-C₁₂-aralkyl. Examples of these hydrocarbon radicals have been mentioned above for the substituents.

Substituted or unsubstituted substituents can, for example, be —CH(O), —C(O)—C₁-C₄-alkyl or —C(O)—C₆-C₁₀-aryl.

Substituted or unsubstituted substituents can, for example, be —CO₂R₀₂ or —C(O)—NR₀₃R₀₄ radicals, where R₀₃, R₀₄ are each, independently of one another, hydrogen, C₁-C₄-alkyl, cyclopentyl, cyclohexyl, phenyl, benzyl or R₀₃ and R₀₄ together form tetramethylene, pentamethylene or 3-oxapentane-1,5-diyl, and R₀₂ is hydrogen, C₁-C₈-alkyl, C₅-C₆-cycloalkyl, phenyl or benzyl.

Substituted or unsubstituted substituents can, for example, be —S(O)—O—R₀₂, —S(O)₂O—R₀₂, —S(O)—NR₀₃R₀₄ or —S(O)₂—NR₀₃R₀₄ radicals, where R₀₃, R₀₄ and R₀₂ are as defined above.

Substituted or unsubstituted substituents can, for example, be —P(OR₀₂)₂ or —P(O)(OR₀₂)₂ radicals, where R₀₂ is as defined above.

Substituted or unsubstituted substituents can, for example, be —P(S)(R₀₂) or —P(S)(OR₀₂)₂ radicals, where R₀₂ is as defined above.

In the case of a preferred group of substituents, these are selected from among C₁-C₄-alkyl, substituted or unsubstituted phenyl, tri(C₁-C₄-alkyl)Si, triphenylsilyl, halogen (in particular F, Cl and Br), —OH, —SH, —OR_{b}, —SR_{b}, —CH₂OH, —CH₂O—R_{b}, —NR₀₃R₀₄, —CH(O), —CO₂H, —CO₂R_{b}, —C(O)—NR₀₃R₀₄, where R_{b} is a hydrocarbon radical having from 1 to 10 carbon atoms and R₀₃ and R₀₄ are each, independently of one another, hydrogen, C₁-C₄-alkyl, phenyl, benzyl or R₀₃ and R₀₄ together form tetramethylene, pentamethylene or 3-oxapentane-1,5-diyl, with hydrocarbon radicals being able to be substituted.

Hydrocarbon radicals as or in substituents can in turn be monosubstituted or polysubstituted, for example monosubstituted to trisubstituted, preferably monosubstituted or disubstituted, by, for example, halogen (F, Cl or Br, in particular F), —OH, —SH, —CH(O), —CN, —NR₀₃R₀₄, —C(O)—O—R₀₂, —S(O)—O—R₀₂, —S(O)₂—O—R₀₂, —P(OR₀₂)₂, —P(O)(OR₀₂)₂, —C(O)—NR₀₃R₀₄, —S(O)—NR₀₃R₀₄, —S(O)₂—NR₀₃R₀₄, —O—(O)C—R₀₅, —R₀₃N(O)—C—R₀₅, —R₀₃N—S(O)—R₀₅, —R₀₃N—S(O)₂—R₀₅, C₁-C₄-alkyl, C₁-C₄-alkoxy, C₁-C₄-alkylthio, C₅-C₆-cycloalkyl, phenyl, benzyl, phenoxy or benzyloxy, where R₀₃ and R₀₄ are each, independently of one another, hydrogen, C₁-C₄-alkyl, cyclopentyl, cyclohexyl, phenyl, benzyl or R₀₃ and R₀₄ together form tetramethylene, pentamethylene or 3-oxapentane-1,5-diyl, R₀₂ is hydrogen, C₁-C₈-alkyl, C₅-C₆-cycloalkyl, phenyl or benzyl, and R₀₅ is C₁-C₁₈-alkyl and preferably C₁-C₁₂-alkyl, C₁-C₄-haloalkyl, C₁-C₄-hydroxyalkyl, C₅-C₈-cycloalkyl (for example cyclopentyl, cyclohexyl), C₆-C₁₀-aryl (for example phenyl or naphthyl) or C₇-C₁₂-aralkyl (for example benzyl).

Examples of substituted or unsubstituted substituents are methyl, ethyl, n- and i-propyl, n-, i- and t-butyl, pentyl, hexyl, cyclohexyl, cyclohexylmethyl, phenyl, benzyl, trimethylsilyl, F, Cl, Br, methylthio, methylsulphonyl, methylsulphoxyl, phenylthio, phenylsulphonyl, phenylsulphoxyl, —CH(O), —C(O)OH, —C(O)—OCH₃, —C(O)—OC₂H₅, —C(O)—NH₂, —C(O)—NHCH₃, —C(O)—N(CH₃)₂, —SO₃H, —S(O)—OCH₃, —S(O)—OC₂H₅, —S(O)₂—OCH₃, —S(O)₂—OC₂H₅, —S(O)—NH₂, —S(O)—NHCH₃, —S(O)—N(CH₃)₂, —S(O)—NH₂, —S(O)₂—NHCH₃, —S(O—N(CH₃)₂, —P(OH)₂, —PO(OH)₂, —P(OCH₃)₂, —P(OC₂H₅)₂, —PO(OCH₃)₂, —PO(OC₂H₅)₂, trifluoromethyl, methylcyclohexyl, methyl-cyclohexylmethyl, methylphenyl, dimethylphenyl, methoxyphenyl, dimethoxyphenyl, hydroxymethyl, β-hydroxyethyl, γ-hydroxypropyl, —CH₂NH₂, —CH₂N(CH₃)₂, —CH₂CH₂NH₂, —CH₂CH₂N(CH₃)₂, methoxymethyl, ethoxymethyl, methoxyethyl, ethoxyethyl, HS—CH₂—, HS—CH₂CH₂—, CH₃S—CH₂—, CH₃S—CH₂CH₂—, —CH₂—C(O)OH, —CH₂CH₂—C(O)OH, —CH₂—C(O)OCH₃, —CH₂CH₂—C(O)OCH₃, —CH₂—C(O)NH₂, —CH₂CH₂—C(O)NH₂, —CH₂C(O)—N(CH₃)₂, —CH₂CH₂—C(O)—N(CH₃)₂, —CH₂—SO₃H, —CH₂CH₂SO₃H, —CH₂—SO₃CH₃, —CH₂CH₂—SO₃CH₃, —CH₂—SO₂NH₂, —CH₂—SO₂N(CH₃)₂, —CH₂—PO₃H₂, —CH₂CH₂—PO₃H₂, —CH₂—PO(OCH₃), —CH₂CH₂—PO(OCH₃)₂, —C₆H₄—C(O)OH, —C₆H₄—C(O)OCH₃, —C₆H₄—S(O)₂OH, —C₆H₄—S(O)₂OCH₃, —CH₂O—C(O)CH₃, —CH₂CH₂—O—C(O)CH₃, —CH₂—NH—C(O)CH₃, —CH₂CH₂—NH—C(O)CH₃, —CH₂—O—S(O)₂CH₃, —CH₂CH₂—O—S(O)₂CH₃, —CH₂—NH—S(O)₂CH₃, —CH₂CH₂—NH—S(O)₂CH₃, —P(O)(C₁-C₈-alkyl)₂, —P(S)(C₁-C₈-Alkyl)₂, —P(O)(C₆-C₁₀-aryl)₂, —P(S)(C₆-C₁₀-aryl)₂, —C(O)—C₁-C₈-alkyl, —C(O)—C₆-C₁₀-aryl, —OH, —SH, —NHCH₃, —N(CH₃)₂, —NH₂, pyrrolidinyl, piperidinyl, morpholinyl, methoxy, ethoxy, and hydroxyethoxy.

For the purposes of the invention, the substituents defined below are preferably substituents as mentioned above including the embodiments and preferences.

To illustrate skeletons of possible anhydrides, the following formulae of cyclic anhydrides are presented by way of example. Examples of cyclic anhydrides having an aliphatic or heteroaliphatic radical are anhydrides of the formulae I and II

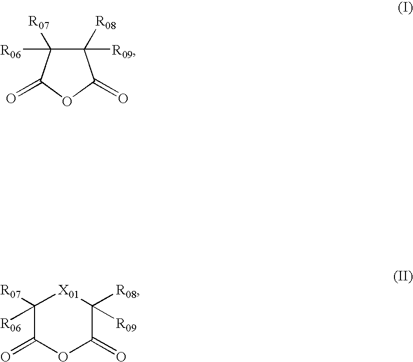

where R₀₆, R₀₇, R₀₈, and R₀₉ are each, independently of one another, hydrogen or a substituent, in the formula I R₀₆ and R₀₉ are as defined above and R₀₇ and R₀₈ represent a bond or R₀₆ and R₀₉ are as defined above and R₀₇ and R₀₈ in the formula I are together —C(O)—O—C(O)—, —O—CR₀₁₀R₀₁₁(O)—, —O—C_{y}H_{2y}O—, —NRₐCR₀₁₀R₀₁₁NRₐ— or —C(O)—C_{y}H_{2y}—(O)—, R₀₆ and R₀₇ or R₀₈ and R₀₉ in each case together form a substituted or unsubstituted tetramethylene or pentamethylene, X₀₁ is —O—, —NH—, —NRₐ— or —C_{y}H_{2y}—, preferably —CR₀₁₀R₀₁₁—, y is from 1 to 5, preferably from 1 to 3 and particularly preferably 1 or 2, R₀₁₀ and R₀₁₁ are each, independently of one another, hydrogen or a substituent, R₀₇ and/or R₀₈ together with a terminal carbon atom of the group —C_{y}H_{2y}— represent a bond or R₀₇ and R₀₁₀ represent a bond. Examples of substituents R₀₆ and R₀₉ are HO—, protected HO—, C₁-C₆-alkoxy, —NR₀₃R₀₄ where R₀₃ and R₀₄ are, independently of one another, hydrogen, C₁-C₆-alkyl, phenyl or benzyl.

Examples of cyclic anhydrides having a cycloaliphatic or heterocycloaliphatic radical are anhydrides of the formula III

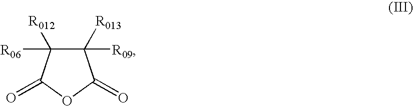

where R₀₆ and R₀₉ are each, independently of one another, hydrogen or a substituent or R₀₆ and R₀₉ together represent a bond and R₀₁₂ and R₀₁₃ together with the carbon atoms to which they are bound form an unsubstituted or substituted monocyclic or polycyclic, saturated or ethylenically unsaturated hydrocarbon ring system and/or heterohydrocarbon ring system containing the groups —O—, S—; —N═, —NH— and —NRₐ—. R₀₁₂ and R₀₁₃ can, for example, together form an unsubstituted or substituted divalent radical selected from the group consisting of —CᵣH₂ᵣ where r is from 2 to 5, —HC═CH—, —HC═CH—CH₂—, —HC═CH—CH₂CH₂, CH₂HC═CH—CH₂—, —HC═CH—CH═CH—, ═CH—CH₂—CH═, ═CH—CH₂—CH₂CH═, —O—CH₂O—, —O—CH(CH₃)—O—, —O—C(CH₃)₂—O—, —CH₂—O—CH₂, —CH₂—O—CH₂—CH₂, —CH₂—O—CH═CH—, —CH₂S—CH₂—, —CH₂—S—CH₂—CH₂—, —CH₂NH—CH₂—, —CH₂—NRₐ—CH₂—, —CH₂—NH—CH₂—CH₂, —CH₂—NRₐ—CH₂—, —CH₂NRₐ—CH₂—CH₂—, —NRₐ—CH(CH)₃—NRₐ—, —NRₐ—C(CH₃)₂—NRₐ— and —NRₐ—CH₂—NRₐ—. In the case of double bonds on the carbon atoms of the anhydride group, R₀₆ and/or R₀₉ represent a bond to the radicals R₀₁₂ and/or R₀₁₃. The H atoms of the divalent radicals can be substituted, and aliphatic and/or aromatic hydrocarbon rings or heterohydrocarbon rings can be fused on, with the aliphatic rings also being able to be bridged.

Examples of cyclic anhydrides having cycloaliphatic-aliphatic and heterocycloaliphatic-aliphatic radicals are anhydrides of the formula IV,

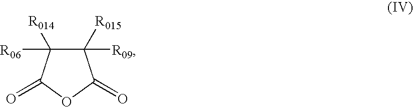

where R₀₆ and R₀₉ are each, independently of one another, hydrogen or a substituent or R₀₆ and R₀₉ together represent a bond and R₀₁₄ and R₀₁₅ together with the carbon atoms to which they are bound form an unsubstituted or substituted monocyclic or polycyclic, saturated or ethylenically unsaturated hydrocarbon ring system and/or heterohydrocarbon ring system containing the groups —O—, S—; —NH—, —N═ and —NRₐ—, with one or both carbonyl groups being bound to the rings via a —CH₂— or —CH₂—CH₂— group. The H atoms of the divalent radicals can be substituted and aliphatic and/or aromatic hydrocarbon rings or heterohydrocarbon rings can be fused on, with the rings also being able to be bridged. R₀₁₄ and R₀₁₅ together can form, for example, cyclobutane, cyclobutene, cyclopentane, cyclopentene, cyclohexane, cyclohexene, cycloheptane, cycloheptene, cycloheptadiene, cyclooctane, cyclooctene, cyclooctadiene, dihydrofuran, tetrahydrofuran, dihydrothiophene and tetrahydrothiophene, pyrroline, pyrrolidine, N-methylpyrroline, N-methylpyrrolidine, piperidine, piperazine, morpholine, dihydrobenzofuran or dihydroindole which contain a bound —CH₂— or —CH₂—CH₂-group or —CH₂— or —CH₂—CH₂— groups bound in the 1,2 positions.

Examples of cyclic anhydrides having aromatic and heteroaromatic radicals are anhydrides of the formula V,

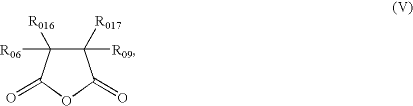

where R₀₆ and R₀₉ together represent a bond and R₀₁₆ and R₀₁₇ together with the carbon atoms to which are bound form an unsubstituted or substituted monocyclic or polycyclic, aromatic or heteroaromatic, five- or 6-membered ring. Aliphatic, bridged or unbridged hydrocarbon rings or heterohydrocarbon rings may be fused onto these rings. Some examples of such rings are phenylene, naphthylene, furan-2,3- or -3,4-diyl, thiophene-2,3- or -3,4-diyl, pyrrole-2,3- or -3,4-diyl, benzofurandiyl, benzothiophenediyl, pyridinylene, pyrimidinylene, pyrazinylene, quinolinediyl.

Examples of cyclic anhydrides having aromatic-aliphatic and heteroaromatic-aliphatic radicals are anhydrides of the formula VI,

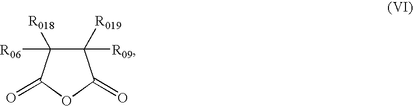

where R₀₆ and R₀₉ together represent a bond and R₀₁₈ and R₀₁₉ together with the carbon atoms to which they are bound form an unsubstituted or substituted monocyclic or polycyclic, aromatic or heteroaromatic, five- or six-membered ring, with one or both carbonyl groups being bound to the rings via a —CH₂— or —CH₂—CH₂— group. Aliphatic, bridged or unbridged hydrocarbon rings or heterohydrocarbon rings may be fused onto these rings. Some examples of such rings are —C₆H₄—CH₂—, —CH₂—C₆H₄—CH₂—, —C₆H₄—CH₂CH₂—, —CH₂—C₅H₃N—, —CH₂—C₄H₃O— and —C₄H₃NH—CH₂—.

The compounds of the formulae III to VI may contain further —C(O)—O—C(O)— groups bound in the 1,2 positions.

Suitable, homogeneous iridium catalysts correspond, for example, to the formulae VIII and VIII,

[A₁Me₁YZ] (VII),

[A₁Me₁Y]⁺E₁⁻ (VIII),

where
A₁ represents two tertiary monophosphines or one ditertiary diphosphine which together with the Ir atom forms a five- to ten-membered, preferably five- to eight-membered and particularly preferably five- to seven-membered ring;
Me₁ is iridium;
Y represents two olefins or one diene;

### Z is Cl, Br or I; and

E₁⁻ is the anion of an oxy acid or complex acid.

Olefins Y can be C₂-C₁₂-, preferably C₂-C₆- and particularly preferably C₂-C₄-olefins. Examples are propene, 1-butene and in particular ethylene. The diene can have from 5 to 12 and preferably from 5 to 8 carbon atoms and can be an open-chain, cyclic or polycyclic diene. The two olefin groups of the diene are preferably connected by one or two CH₁— groups. Examples are 1,3-pentadiene, cyclopentadiene, 1,5-hexadiene, 1,4-cyclohexadiene, 1,4- or 1,5-heptadiene, 1,4- or 1,5-cycloheptadiene, 1,4- or 1,5-octadiene, 1,4- or 1,5-cyclooctadiene and norbornadiene. Y preferably represents two ethylenes or 1,5-hexadiene, 1,5-cyclooctadiene or norbornadiene.

In the formula VII, Z is preferably Cl or Br. Examples of E₁⁻ are ClO₄⁻, CF₃SO₃⁻, CH₃SO₃⁻, HSO₄⁻, BF₄⁻, B(phenyl)₄⁻, B(3,5-bistrifluoromethylphenyl)₄⁻, PF₆⁻, SbCl₆⁻, AsF₆⁻ or SbF₆⁻.

For the purposes of the invention, tertiary monophosphines are ligands in which three O-bonded substituents are bound to one P atom (phosphites) two O-bonded substituents and one N-bonded substituent are bound to one P atom or three C-bonded substituents are bound to one P atom. For the purposes of the invention, ditertiary diphosphines are ligands in which two P atoms are joined via a bridging group and the P atoms are bound to the bridging group via O, N or C atoms and the P atoms further bear two O- or C-bonded substituents. Suitable substituents are described and explained below for tertiary monophosphines having three C-bonded substituents, and also for phosphino groups X₁ and X₂. Two O-bonded substituents preferably represent the radical of a diol, so that a cyclic phosphonite group is present. Diols are preferably 2,2′-dihydroxy-1,1′-biphenyls or -binaphthyls which may be monosubstituted or polysubstituted, in particular in the 6 and/or 6′ positions, for example by C₁-C₈-alkyl, C₅-C₈-cycloalkyl, C₅-C₈-cycloalkyl-C₁-C₄-alkyl, C₆-C₁₀-aryl, C₇-C₁₂-aralkyl, C₁-C₈-alkyloxy, C₅-C₈-cycloalkyloxy, C₅-C₈-cycloalkyl-C₁-C₄-alkyloxy, C₆-C₁₀-aryloxy or C₇-C₁₂-aralkyloxy. Some examples are methyl, ethyl, propyl, butyl, cyclohexyl, cyclohexylmethyl, phenyl, benzyl, methoxy, ethoxy, propoxy, butoxy, cyclohexyo, phenyloxy and benzyloxy.

A large number of tertiary monophosphines and tertiary diphosphines are known and described in the literature. Monophosphines and diphosphines can be chiral in order to induce the predominant formation of optical isomers in the hydrogenation of prochiral cyclic anhydrides.

Suitable tertiary monophosphines having three O-bonded or two O-bonded and one N-bonded substituents can, for example, have the formulae

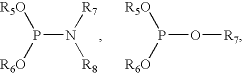

where
R₅, R₆, R₇ and R₈ are each, independently of one another, a monovalent, unsubstituted or substituted aliphatic, heteroaliphatic, cycloaliphatic, heterocycloaliphatic, cycloaliphatic aliphatic, heterocycloaliphatic-aliphatic, aromatic, heteroaromatic, aromatic-aliphatic or heteroaromatic-aliphatic radical, R₅ and R₆ together form a divalent, unsubstituted or substituted aliphatic, heteroaliphatic, cycloaliphatic, heterocycloaliphatic, cycloaliphatic aliphatic, heterocycloaliphatic-aliphatic, aromatic, heteroaromatic, aromatic-aliphatic or heteroaromatic-aliphatic radical and R₇ and R₈ together with the N atom form a 5- or 6-membered ring. Suitable substituents have been described above and embodiments and preferences for monovalent and divalent radicals are mentioned below for phosphines having C-bonded substituents. R₅ and R₆ preferably together form a divalent radical, particularly preferably unsubstituted or substituted 1,1′-binaphth-2,2′-diyl or 1,1′-biphen-2,2′-diyl. Examples of the latter are ligands of the formulae

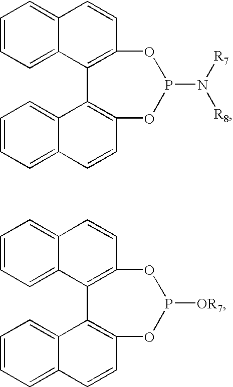

where R₇ and R₈ are as defined above.

Suitable tertiary monophosphines contain three C-bonded substituents from the group consisting of unsubstituted or substituted aliphatic, heteroaliphatic, cycloaliphatic, heterocycloaliphatic, cylcoaliphatic-aliphatic, heterocycloaliphatic-aliphatic, aromatic, heteroaromatic, aromatic-aliphatic and heteroaromatic-aliphatic radicals which, for example, contain a total of from 1 to 18, preferably from 1 to 12 and particularly preferably from 1 to 8, carbon and/or heteroatoms and from 4 to 8, preferably from 5 to 7 and particularly preferably 5 or 6, ring atoms. The cyclic radicals mentioned can be bridged, fused or bridged and fused to form polycyclic radicals. Such ring systems can, for example, comprise from 2 to 6 and preferably from 2 to 4 cyclic or heterocyclic hydrocarbon radicals. Heteroatoms in heteroradicals can, for example, be selected from the group consisting of —O—, —S—, ═N—, —HN— and —RₐN—, where Rₐ is preferably C₁-C₈-alkyl, C₅-C₈-cycloalkyl, C₅-C₈-cycloalkyl-C₁-C₄-alkyl, C₆-C₁₀-aryl, C₇-C₁₂-aralkyl or C₁-C₈-acyl. Aliphatic or heteroaliphatic radicals can be, for example, linear or branched C₁-C₁₂alkyl and preferably C₃-C₈-alkyl which may be interrupted by —O— or —S—. Cycloaliphatic or heterocycloaliphatic radicals can be, for example, C₅-C₈-cycloalkyl which may be interrupted by —O—, —S— or —NRₐ—. Cycloaliphatic-aliphatic or heterocycloaliphatic-aliphatic radicals can be, for example, C₅-C₈-cycloalkyl-C₁-C₄-alkyl whose ring may be interrupted by —O—, —S— or —NRₐ—. Aromatic or heteroaromatic radicals can be, for example, C₆-C₁₂-aryl or C₅-C₁₁-heteroaryl containing heteroatoms selected from the group consisting of —O—, —S—, ═N—, —HN— and —RₐN—. Aromatic-aliphatic or heteroaromatic-aliphatic radicals can be, for example, C₆-C₁₂-aryl-C₁-C₄-alkyl or C₅-C₁₁-heteroaryl-C₁-C₄-alkyl containing heteroatoms selected from the group consisting of —O—, —S—, ═N—, —HN— and —RₐN—. Tertiary phosphines can also be P-substituted P-cyclic rings having, for example, a total of from 4 to 6 ring atoms (phosphetanes, phospholanes, phosphanes). The P substituents of the tertiary phosphines can be substituted, for example as described below for ditertiary diphosphines. The tertiary diphosphines can bear identical or different substituents. Some examples of tertiary monophosphines are trimethylphosphine, tri-t-butylphosphine, trihexylphosphine, tricyclohexylphosphine, trinorbornylphosphine, triadamantylphosphine, triphenylphosphine, tritolylphosphine, trixylylphosphine, phenylphospholane and diphenyl-t-butylphosphine.

The achiral and chiral ditertiary diphosphines can be diphosphines in which the two phosphine groups are bound to different carbon atoms of linear or cyclic bridging groups, preferably
(a) bound to different carbon atoms of a carbon chain having from 2 to 6 carbon atoms, where the carbon chain may be part of a monocyclic ring or part of a bicyclic ring system (for example biphenyl, binaphthyl, or cyclopentadienylphenyl, cyclopentadienyl-CH₂-phenyl, cyclopentadienyl-CH(OCH₃)-phenyl in ferrocenes) or
(b) bound in each case to a cyclopentadienyl ring of a substituted or unsubstituted ferrocene.

The ditertiary diphosphines contain two secondary phosphino groups X₁ and X₂, which can bear two identical or two different hydrocarbon radicals. The secondary phosphino groups X₁ and X₂ preferably each bear two identical hydrocarbon radicals. Furthermore, the secondary phosphino groups X₁ and X₂ can be identical or different.

The hydrocarbon radicals can be unsubstituted or substituted and/or contain heteroatoms selected from the group consisting of O, S and N. They can have from 1 to 22, preferably from 1 to 12 and particularly preferably from 1 to 8, carbon atoms. A preferred secondary phosphine is any one in which the phosphino group bears two identical or different radicals selected from the group consisting of linear or branched C₁-C₁₂-alkyl; unsubstituted or C₁-C₆-alkyl or C₁-C₆-alkoxy-substituted, C₅-C₁₂-cycloalkyl or C₅-C₁₂-cycloalkyl-CH₂; phenyl, naphthyl, furyl or benzyl; and phenyl or benzyl substituted by halogen (for example F, Cl and Br), C₁-C₆-alkyl, C₁-C₆-haloalkyl (for example trifluoromethyl), C₁-C₆-alkoxy, C₁-C₆-haloalkoxy (for example trifluoromethoxy), (C₆H₅)₃Si, (C₁-C₁₂alkyl)₃Si, secondary amino or —CO₂—C₁-C₆-alkyl (for example —CO₂CH₃).

Examples of alkyl substituents on P, which preferably contain from 1 to 6 carbon atoms, are methyl, ethyl, n-propyl, i-propyl, n-butyl, i-butyl, t-butyl and the isomers of pentyl and hexyl. Examples of unsubstituted or alkyl-substituted cycloalkyl substituents on P are cyclopentyl, cyclohexyl, methyl cyclohexyl and ethylcyclohexyl, and dimethylcyclohexyl. Examples of alkyl-, alkoxy-, haloalkyl-, haloalkoxy- and halogen-substituted phenyl and benzyl substituents on P are o-, m- or p-fluorophenyl, o-, m- or p-chlorophenyl, difluorophenyl or dichlorophenyl, pentafluorophenyl, methylphenyl, dimethylphenyl, trimethylphenyl, ethylphenyl, methylbenzyl, methoxyphenyl, dimethoxyphenyl, trifluoromethylphenyl, bis-trifluoromethylphenyl, tris-trifluoromethylphenyl, trifluoromethoxyphenyl, bis-trifluoromethoxyphenyl and 3,5-dimethyl-4-methoxyphenyl.

Preferred secondary phosphino groups are those in which the identical radicals are selected from the group consisting of C₁-C₆-alkyl, unsubstituted cyclopentyl or cyclohexyl and cyclopentyl or cyclohexyl substituted by from 1 to 3 C₁-C₄-alkyl or C₁-C₄-alkoxy groups, benzyl and in particular phenyl which are unsubstituted or substituted by from 1 to 3 C₁-C₄-alkyl, C₁-C₄-alkoxy, F, Cl, C₁-C₄-fluoroalkyl or C₁-C₄-fluoroalkoxy groups.

The secondary phosphino group preferably corresponds to the formula —PR₁R₂, where R₁ and R₂ are each, independently of one another, a hydrocarbon radical which has from 1 to 18 carbon atoms and is unsubstituted or substituted by halogen, C₁-C₆-alkyl, C₁-C₆-haloalkyl, C₁-C₆-alkoxy, C₁-C₆-haloalkoxy, (C₁-C₄-alkyl)₂-amino, (C₆H₅)₃Si, (C₁-C₁₂-alkyl)₃Si, or —CO₂—C₁-C₆-alkyl, and/or contains heteroatoms O.

R₁ and R₂ are preferably identical radicals selected from the group consisting of linear or branched C₁-C₆-alkyl, unsubstituted cyclopentyl or cyclohexyl and cyclopentyl or cylcohexyl substituted by from one to three C₁-C₄-alkyl or C₁-C₄-alkoxy groups, furyl, unsubstituted benzyl or benzyl substituted by from one to three C₁-C₄-alkyl or C₁-C₄-alkoxy groups and in particular unsubstituted phenyl or phenyl substituted by from one to three C₁-C₄-alkyl, C₁-C₄-alkoxy, —NH₂, —N(C₁-C₆-alkyl)₂, OH, F, Cl, C₁-C₄-fluoroalkyl or C₁-C₄-fluoroalkoxy groups,

R₁ and R₂ are particularly preferably identical radicals selected from the group consisting of C₁-C₆-alkyl, cyclopentyl, cyclohexyl, furyl, and unsubstituted phenyl or phenyl substituted by from one to three C₁-C₄-alkyl, C₁-C₄-alkoxy and/or C₁-C₄-fluoroalkyl groups,

The secondary phosphino groups X₁ and X₂ can be cyclic secondary phosphino, for example groups of the formulae

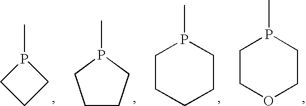

which are unsubstituted or substituted by one or more —OH, C₁-C₈-alkyl, C₄-C₈-cycloalkyl, C₁-C₆-alkoxy, C₁-C₄-alkoxy-C₁-C₄-alkyl, phenyl, C₁-C₄-alkylphenyl or C₁-C₄-alkoxyphenyl, benzyl, C₁-C₄-alkylbenzyl or C₁-C₄-alkoxybenzyl, benzyloxy, C₁-C₄-alkylbenzyloxy or C₁-C₄-alkoxybenzyloxy, or C₁-C₄-alkylidenedioxyl groups.

The substituents can be bound to the P atom in one or both α positions in order to introduce chiral carbon atoms. Substituents in one or both α positions are preferably C₁-C₄-alkyl or benzyl, for example methyl, ethyl, n- or i-propyl, benzyl or —CH₂—O—C₁-C₄-alkyl or —CH₂—O—C₆-C₁₀-aryl.

Substituents in the β,γ positions can be, for example, C₁-C₄-alkyl, C₁-C₄-alkoxy, benzyloxy, or —O—CH₂—O—, —O—CH(C₁-C₄-alkyl)-O— and —O—C(C₁-C₄-alkyl)₂-O—. Some examples are methyl, ethyl, methoxy, ethoxy, —O—CH(methyl)-O— and —O—C(methyl)₂-O—.

Other known and suitable secondary phosphino radicals are those derived from cyclic and chiral phospholanes having seven carbon atoms in the ring, for example those of the formulae

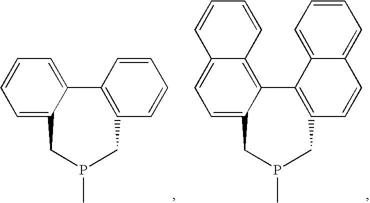

where the aromatic rings may be substituted by C₁-C₄-alkyl, C₁-C₄-alkoxy, C₁-C₄-alkoxy-C₁-C₂-alkyl, phenyl, benzyl, benzyloxy or C₁-C₄-alkylidenedioxyl or C₁-C₄-alkylenedioxyl (cf. US 2003/0073868 A1 and WO 02/048161).

Depending on the type of substitution and number of substituents, the cyclic phosphino radicals can be C-chiral, P-chiral or C- and P-chiral.

An aliphatic 5- or 6-membered ring or benzene can be fused onto two adjacent carbon atoms in the radicals of the above formulae.

The cyclic secondary phosphino can, for example, correspond to the formulae (only one of the possible diastereomers shown),

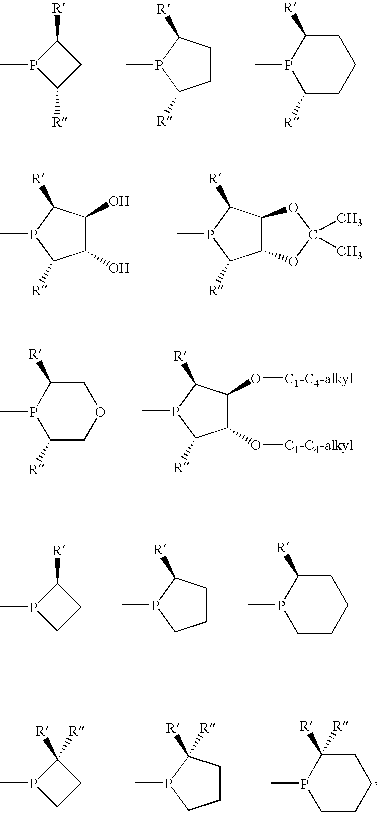

where
the radicals R′ and R″ are each C₁-C₄-alkyl, for example methyl, ethyl, n- or i-propyl, benzyl, or —CH₂—O—C₁-C₄-alkyl or —CH₂—O—C₆-C₁₀-aryl and R′ and R″ are identical or different. When R′ and R″ are bound to the same carbon atom, they can together also form C₄-C₅-alkylene.

In a preferred embodiment, X₁ and X₂ are preferably identical or different acyclic secondary phosphino selected from the group consisting of —P(C₁-C₆-alkyl), —P(C₅-C₈-cycloalkyl)₂, —P(C₇-C₈-bicycloalkyl)₂, —P(o-furyl)₂, —P(C₆H₅)₂, —P[2-(C₁-C₆-alkyl)C₆H₄]₂, —P[3-(C₁-C₆-alkyl)C₆H₄]₂, —P[4-(C₁-C₆-alkyl)C₆H₄]₂, —P[2-(C₁-C₆-alkoxy)C₆H₄]₂, —P[3-(C₁-C₆-alkoxy)C₆H₄]₂, —P[4-(C₁-C₆-alkoxy)C₆H₄]₂, —P[2-(trifluoromethyl)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(C₁-C₆-alkyl)₂C₆H₃]₂, —P[3,5-bis(C₁-C₆-alkoxy)₂C₆H₃]₂ and —P[3,5-bis(C₁-C₆-alkyl)₂-4-(C₁-C₆-alkoxy)C₆H₂]₂ or cyclic phosphino selected from the group consisting of

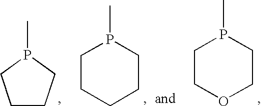

which are unsubstituted or substituted by one or more C₁-C₄-alkyl, C₁-C₄-alkoxy, C₁C₄-alkoxy-C₁-C₂-alkyl, phenyl, benzyl, benzyloxy or C₁-C₄-alkylidenedioxyl groups.

Some specific examples are —P(CH₃)₂, —P(i-C₃H₇)₂, —P(n-C₄H₉)₂, —P(i-C₄H₉), —P(C₆H₁₁)₂, —P(norbornyl)₂, —P(o-furyl)₂, —P(C₆H₅)₂, P[2-(methyl)C₆H₄]₂, P[3-(methyl)C₆H₄]₂, —P[4-(methyl)C₆H₄]₂, —P[2-(methoxy)C₆H₄]₂, —P[3-(methoxy)C₆H₄]₂, —P[4-(methoxy)C₆H₄]₂, —P[3-(tri-fluoromethyl)C₆H₄]₂, —P[4-(trifluoromethyl)C₆H₄]₂, —P[3,5-bis(trifluoromethyl)C₆H₃]₂, —P[3,5-bis-(methyl)₂C₆H₃]₂, —P[3,5-bis(methoxy)₂C₆H₃]₂ and —P[3,5-bis(methyl)₂-4-(methoxy)C₆H₂]₂ and groups of the formulae

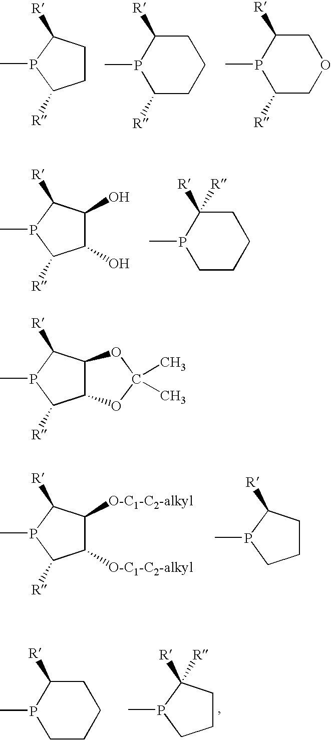

where
R′ is methyl, ethyl, methoxy, ethoxy, phenoxy, benzyloxy, methoxymethyl, ethoxymethyl or benzyloxymethyl and R″ has the same meanings as R′.

The ditertiary diphosphines preferably correspond to the formula IX,

X₁—R₃—X₂ (IX),

where R₃ is unsubstituted or C₁-C₆-alkyl-, C₁-C₆-alkoxy-, C₅- or C₆-cycloalkyl-, phenyl-, naphthyl- or benzyl-substituted C₂-C₄-alkylene; unsubstituted or C₁-C₆-alkyl-, phenyl- or benzyl-substituted 1,2- or 1,3-cycloalkylene, 1,2- or 1,3-cycloalkenylene, 1,2- or 1,3-bicycloalkylene or 1,2- or 1,3-bicycloalkenylene having from 4 to 10 carbon atoms; unsubstituted or C₁-C₆-alkyl-, phenyl- or benzyl-substituted 1,2- or 1,3-cycloalkylene, 1,2- or 1,3-cycloalkenylene, 1,2- or 1,3-bicycloalkylene or 1,2- or 1,3-bicycloalkenylene which has from 4 to 10 carbon atoms and has methylene or C₂-C₄-alkylidene bound to its 1 and/or 2 positions or to its 3 position; 1,4-butylene which is substituted in the 2,3 positions by R_{b}R_{c}C(O—)₂ and in the 1 and/or 4 positions is unsubstituted or substituted by C₁-C₆-alkyl, phenyl or benzyl where R_{b} and R_{c} are each, independently of one another, hydrogen, C₁-C₆-alkyl, phenyl or benzyl; 3,4- or 2,4-pyrrolidinylene or methylene-4-pyrrolidin-4-yl, whose N atom is substituted by hydrogen, C₁-C₁₂-alkyl, phenyl, benzyl, C₁-C₁₂-alkoxycarbonyl, C₁-C₈-acyl, C₁-C₁₂-alkylaminocarbonyl; or unsubstituted or halogen-, HO—, C₁-C₆-alkyl-, C₁-C₆-alkoxy-, phenyl-, benzyl-, phenyloxy- or benzyloxy-substituted 1,2-phenylene, 2-benzylene, 1,2-xylylene, 1,8-naphthylene, 1,1′-binaphthylene or 1,1′-biphenylene; or R₃ is a radical of one of the formulae

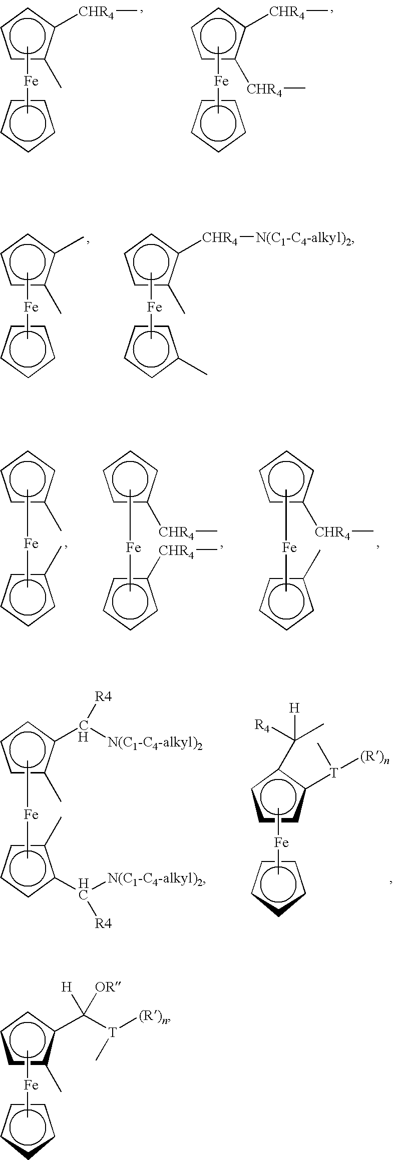

where R₄ is hydrogen, C₁-C₈-alkyl, C₁-C₄-fluoroalkyl, unsubstituted phenyl or phenyl substituted by from 1 to 3 F, Cl, Br, C₁-C₄-alkyl, C₁-C₄-alkoxy or fluoromethyl groups,
n is 0 or an integer from 1 to 4 and R′ are identical or different substituents selected from the group consisting of C₁-C₄-alkyl, C₁-C₄-fluoroalkyl and C₁-C₄-alkoxy;
T is C₆-C₂₀-arylene or C₃-C₁₆-heteroarylene;
the free bond is located in the ortho position relative to the T-cyclopentadienyl bond,
R″ is hydrogen, R₀₀₁R₀₀₂R₀₀₃Si-, halogen-, hydroxy-, C₁-C₈-alkoxy- or R₀₀₄R₀₀₅N-substituted C₁-C₁₈-acyl, or is R₀₀₆—X₀₀₁—C(O)—;
R₀₀₁, R₀₀₂ and R₀₀₃ are each, independently of one another, C₁-C₁₂-alkyl, unsubstituted or C₁-C₄-alkyl- or C₁-C₄-alkoxy-substituted C₆-C₁₀-aryl or C₇-C₁₂-aralkyl;
R₀₀₄ and R₀₀₅ are each, independently of one another, hydrogen, C₁-C₁₂-alkyl, C₃-C₈-cycloalkyl, C₆-C₁₀-aryl or C₇-C₁₂-aralkyl or R₀₀₄ and R₀₀₅ together form trimethylene, tetramethylene, pentamethylene or 3-oxapentylene;
R₀₀₆ is C₁-C₁₈-alkyl, unsubstituted or C₁-C₄-alkyl- or C₁-C₄-alkoxy-substituted C₃-C₈-cycloalkyl, C₆-C₁₀-aryl or C₇-C₁₂-aralkyl; and

### X₀₀₁ is —O— or —NH—.

The cyclopentadienyl rings in the above formulae may be substituted independently of one another, for example by C₁-C₄-alkyl. The tertiary monophosphines and ditertiary diphosphines can be used in the form of racemates, mixtures of diastereomers or in essentially enantiomerically pure form.

A preferred group of achiral and chiral diphosphines are those of the formulae X to XXIX,

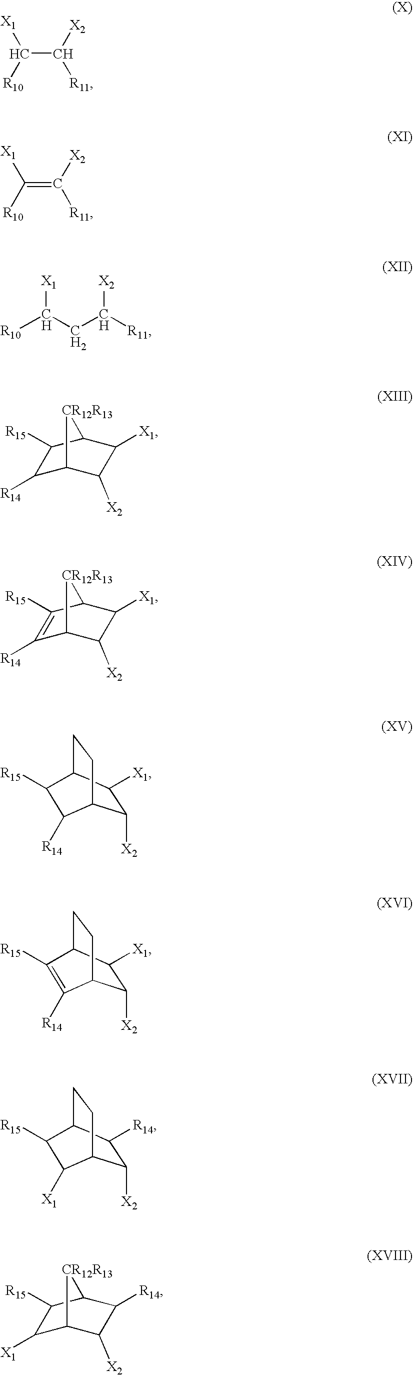

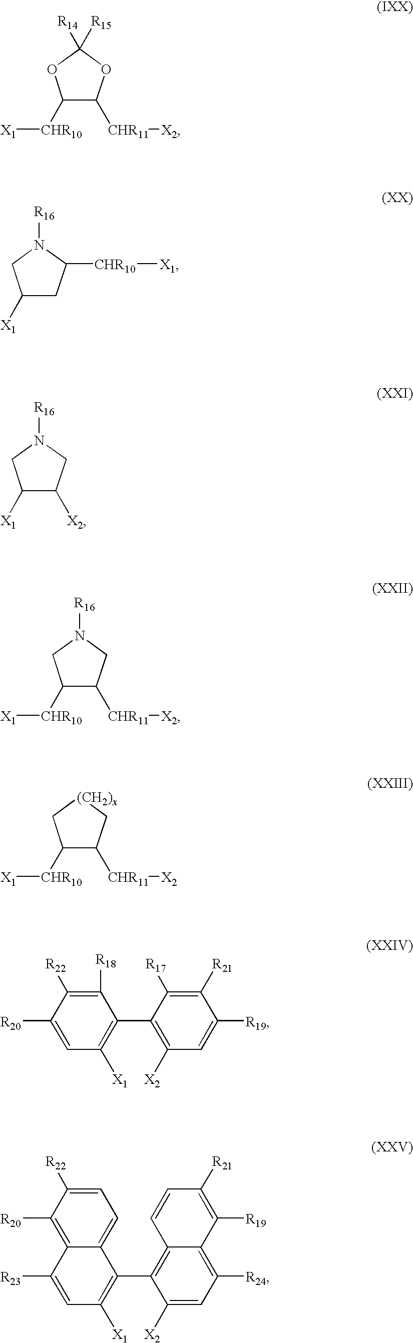

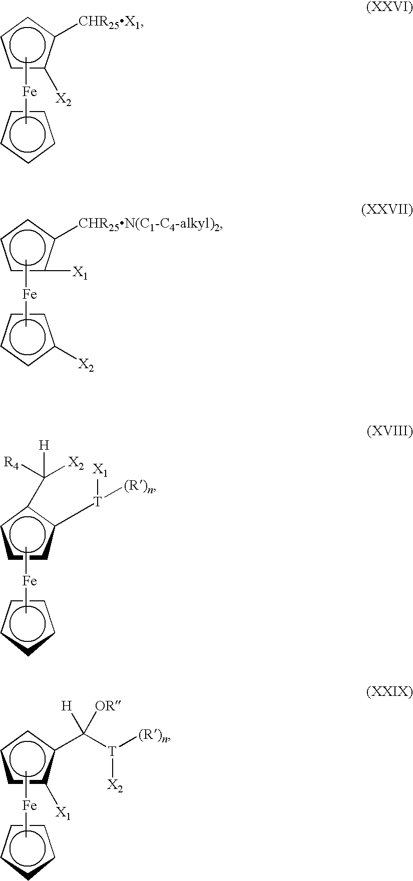

where R₄, T, R′, R″, X₁ and X₂ have the meanings given above, including the preferences, R₁₀ and R₁₁ are each, independently of one another, hydrogen, C₁-C₄-alkyl or unsubstituted benzyl or phenyl or benzyl or phenyl substituted by from one to three C₁-C₄-alkyl or C₁-C₄-alkoxy groups,
R₁₂ and R₁₃ are each, independently of one another, hydrogen C₁-C₄-alkyl, phenyl or benzyl,
R₁₄ and R₁₅ are each, independently of one another hydrogen, C₁-C₄-alkyl, C₁-C₄-alkoxy or unsubstituted benzyl or phenyl or benzyl or phenyl substituted by from one to three C₁-C₄-alkyl or C₁-C₄-alkoxy groups,
R₁₆ is hydrogen, C₁-C₁₂-alkyl, unsubstituted benzyl or phenyl or benzyl or phenyl substituted by from one to three C₁-C₄-alkyl or C₁-C₄-alkoxy groups, C₁-C₁₂-akoxy-C(O)—, unsubstituted phenyl-C(O)— or benzyl-C(O)— or phenyl-C(O)— or benzyl-C(O)— substituted by from one to three C₁-C₄-alkyl or C₁-C₄-alkoxy groups, C₁-C₁₂-alkyl-NH—C(O) or unsubstituted phenyl-NH—C(O)— or benzyl-NH—C(O)— or phenyl-NH—C(O)— or benzyl-NH—C(O)— substituted by from one to three C₁-C₄-alkyl or C₁-C₄-alkoxy groups,
n is 0, 1 or 2,
R₁₇ and R₁₈ are each C₁-C₄-alkyl or C₁-C₄-alkoxy or R₁₇ and R₁₈ together form oxadimethylene,
R₁₉, R₂₀, R₂1, R₂₂, R₂₃ and R₂₄ are each, independently of one another, H, C₁-C₄-alkyl, C₁-C₄-alkoxy, C₅- or C₆-cycloalkyl or -alkoxy, phenyl, benzyl, phenoxy, benzyloxy, halogen, OH, —(CH₂)₃—C(O)—O—C₁-C₄-alkyl, —(CH₂)₃—C(O)—N(C₁-C₄-alkyl)₂ or —N(C₁-C₄-alkyl)₂, or R₁₉ and R₂₁, and/or R₁₇ and R₂₁, and/or R₂₀ and R₂₂, and/or R₁₈ and R₂₂, or R₂₁ and R₂₃ and/or R₂₂ and R₂₄ in each case together form a fused-on 5- or 6-membered, monocyclic or bicyclic hydrocarbon ring system, and
R₂₅ is hydrogen, C₁-C₆-alkyl, cyclohexyl or phenyl.

Some preferred examples of chiral ditertiary disphosphines are those of the following formulae:

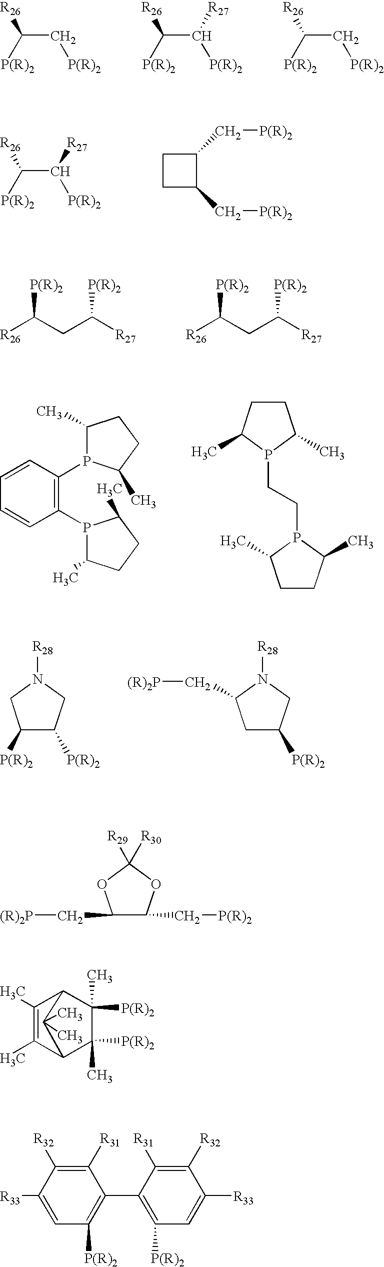

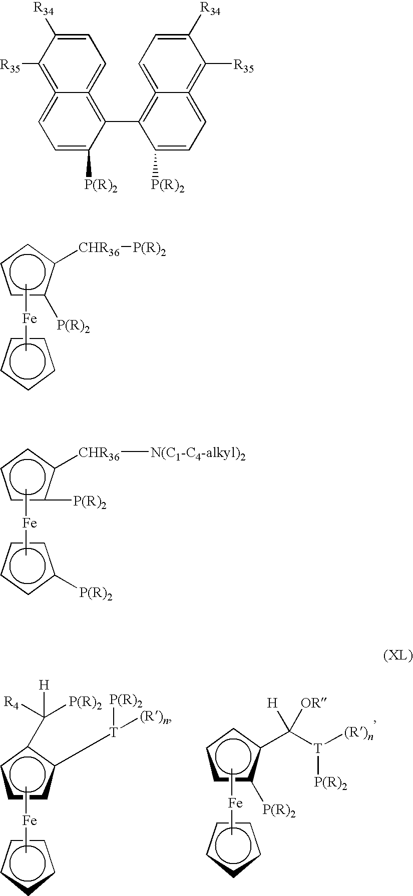

where
R is branched C₁-C₈-alkyl, cyclohexyl, norbornyl, adamantly or unsubstituted phenyl or phenyl substituted by from one to three C₁-C₄-alkyl, C₁-C₄-alkoxy or trifluoromethyl groups or by one —NH₂, (C₁-C₄-alkyl)NH— or (C₁-C₄-alkyl)₂N— group,
R₄ is hydrogen or C₁-C₄-alkyl,
T is 1,2-phenylene, R′ is hydrogen, R″ is C₁-C₄-alkyl,
R₂₆ and R₂₇ are each, independently of one another, C₁-C₄-alkyl, phenyl or benzyl and particularly preferably methyl,
R₂₈ is C₁-C₈-alkyl, C₁-C₈-acyl or C₁-C₈-alkoxycarbonyl,
R₂₉ is hydrogen or independently has one of the meanings given for R₃₀ and R₃₀ is C₁-C₄-alkyl, phenyl or benzyl,
R₃₁ is methyl, methoxy or the two radicals R₃₁ together form oxadimethylene,
R₃₂ and R₃₃ are each, independently of one another, H, C₁-C₄-alkyl, C₁-C₄-alkoxy or (C₁-C₄-alkyl)₂N—,
R₃₄ and R₃₅ are each, independently of one another, H, C₁-C₄-alkyl, C₁-C₄-alkoxy, —(CH₂)₃—C(O)—O—C₁-C₄-alkyl, —(CH₂)₃—C(O)—N(C₁-C₄-alkyl)₂ and
R₃₆ is C₁-C₄-alkyl and particularly preferably methyl.

Suitable ditertiary diphosphines having heterocyclic skeletons are described in EP-A-0 770 085, by T. Benincori et al. in J. of Organomet. Chem. 529 (1997), pages 445 to 453, and in J. Org. Chem., 61, p. 6244, (1996), by F. Bonifacio et al. in Chiratech 1997, Nov. 11 to 13, 1997, Philadelphia, Pa., USA and by L. F. Tietze et al, Chem. Commun. pp. 1811-1812 (1999). Some examples are

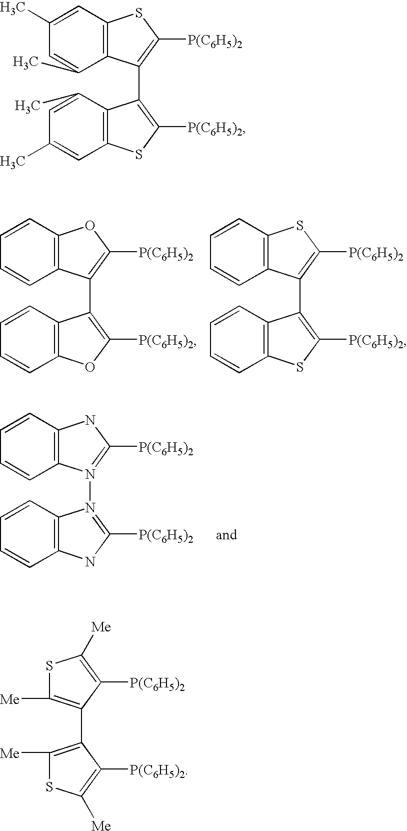

The suitable ditertiary diphosphines are, for example, described in Comprehensive Asymmetric Catalysis (E. N. Jacobsen, A. Pfaltz and H. Yamamoto (eds.), Vol. I-III, Springer Verlag, Berlin, 1999.

The hydrogenation is chemoselective and can therefore be carried out in the presence of other hydrogenatable groups such as carbon double and triple bonds, keto and aldehyde groups. The reaction times are short and high chemical yields are obtained. When use is made of iridium catalysts having chiral ligands, high stereoselectivities can also be achieved when prochiral cyclic anhydrides are employed. During the hydrogenation, it can be observed that a carbonyl group is hydrogenated to the —CH—OH group in a first step. This monohydroxy product can also be detected chromatographically as by-product in the purification of the reaction product.

The metal complexes can be prepared by methods known from the literature (cf. U.S. Pat. No. 5,371,256, U.S. Pat. No. 5,446,844, U.S. Pat. No. 5,583,241 and E. Jacobsen, A. Pfaltz, H. Yamamoto (Eds.), Comprehensive Asymmetric Catalysis I-III, Springer Verlag, Berlin, 1999, and references cited therein).

The following examples illustrate the invention. The ligands L1 to L5 correspond to the following formulae (CH₃ is bound to each of the free bonds):

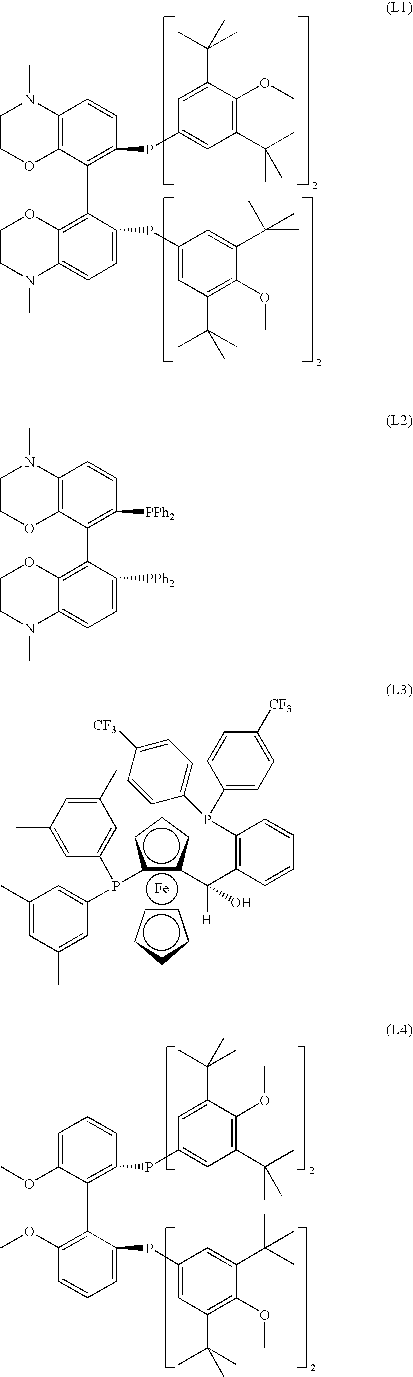

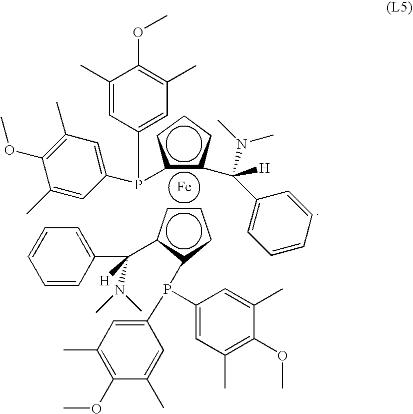

### EXAMPLE 1

### Hydrogenation of cis-cyclohexane-1,2-dicarboxylic anhydride

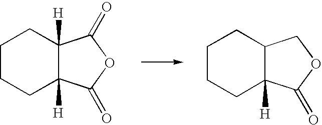

0.25 g (1.62 mmol) of cis-cyclohexane-1,2-dicarboxylic anhydride and 5 ml of degassed dichloromethane are introduced in succession into a Schlenk vessel filled with argon. In a second Schlenk vessel filled with argon, a catalyst solution comprising 10.9 (0.0165 mmol) of [Ir(cyclo-1,5-octadiene)Cl]₂, 30.5 mg (0.036 mmol) (R)-2,2′-diphenylphosphino-6,6′-dimethoxy-1,1′-biphenyl and 5 ml of degassed dichloromethane is prepared. This solution and the catalyst solution are then transferred in succession by means of a steel capillary into a 50 ml steel autoclave filled with argon. The s/c (substrate to catalyst) ratio is 50. The autoclave is closed and a pressure of 80 bar is set by means of 4 flushing cycles (pressurization to 20 bar of hydrogen). The autoclave is heated to 60° C. and the reaction is started by switching on the stirrer. The reactor is stirred for 15 hours. After cooling and opening the reactor, a reddish reaction solution is isolated. The conversion is quantitative (determined by means of GC and ¹H-NMR). Removal of the solvent on a rotary evaporator gives a quantitative yield of the lactone having an enantiomeric purity of 62% ee (ee=in enantiomeric excess) (determined by means of GC; column: Beta-Dex.).

### EXAMPLE 2

### Preparation of 3-methyl-δ-valerolactone of the formula

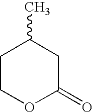

Under an argon atmosphere, 6.72 mg (0.01 mmol) of [Ir(1,5-cyclooctadiene)Cl]₂ and 25.94 mg (0.021 mmol) of (R)-L1 and 5 ml of tetrahydrofuran are introduced in succession into a 10 ml Schlenk vessel. This solution is stirred at room temperature for 15 minutes. 256.26 mg (2 mmol) of racemic 3-methylglutaric anhydride and 5 ml of tetrahydrofuran are introduced under an argon atmosphere into a second Schlenk vessel. The substrate solution is stirred for 5 minutes. The anhydride/Ir ratio is 100. The catalyst solution and the substrate solution are subsequently transferred in succession by means of a steel capillary into a 50 ml steel autoclave under an argon atmosphere. The autoclave is closed, and the argon atmosphere is replaced by a hydrogen atmosphere in 4 flushing cycles (20 bar/1 bar). The hydrogen pressure is set to 70 bar and the autoclave is heated to 60° C. while stirring. After 15 hours, the autoclave is cooled and depressurized. The conversion and the enantiomeric purity are determined by gas chromatography. The conversion is 100% and the enantiomeric purity of the 3-methyl-δ-valerolactone formed is 51% ee.

### EXAMPLE 3

### Preparation of 3-methyl-δ-valerolactone

The procedure of Example 2 is repeated using 14.02 mg (0.021 mmol) of (R)-L2 in place of (R)-L1. The hydrogen pressure is 70 bar, and the reaction temperature is 110° C. The conversion is 74%, and the ee is 16%.

### EXAMPLE 4

### Preparation of 3-oxabicyclo[3.1.0]hexan-2-one of the formula

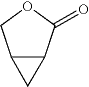

Under an argon atmosphere, 13.43 mg (0.02 mmol) of [Ir(1,5-cyclooctadiene)Cl]₂ and 25.94 mg (0.042 mmol) of (S)—(S)-L3 and 5 ml of tetrahydrofuran are introduced in succession into a 10 ml Schlenk vessel. This solution is stirred at room temperature for 15 minutes. 222.16 mg (2 mmol) of cis-3-oxabicyclo[3.1.0]hexane-2,4-dione and 5 ml of tetrahydrofuran are introduced under an argon atmosphere into a second Schlenk vessel. The substrate solution is stirred for 5 minutes. The anhydride/Ir ratio is 50. The catalyst solution and the substrate solution are subsequently transferred in succession by means of a steel capillary into a 50 ml steel autoclave filled with an argon atmosphere. The autoclave is closed, and the argon atmosphere is replaced by a hydrogen atmosphere in 4 flushing cycles (20 bar/1 bar). The hydrogen pressure is set to 80 bar and the autoclave is heated to 100° C. while stirring. After 20 hours, the autoclave is cooled and depressurized. The conversion and the enantiomeric purity are determined by gas chromatography. The conversion is 62% and the enantiomeric purity of the bicyclic γ-lactone formed (3-oxabicyclo[3.1.0]hexan-2-one) is 21% ee.

### EXAMPLE 5

### Preparation of 3-oxabicyclo[3.1.0]hexan-2-one

The procedure of Example 4 is repeated using 13.43 mg (0.021 mmol) of (R)-2,2′-diphenylphosphino-6,6′-dimethoxy-1,1′-biphenyl in place of (S)—(S)-L3. The hydrogen pressure is 80 bar, and the reaction temperature is 100° C. The conversion is 92%, and the ee is 24%.

### EXAMPLE 6

### Preparation of 3-oxabicyclo[3.4.0]nonan-2-one of the formula

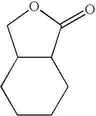

Under an argon atmosphere, 6.74 mg (0.01 mmol) of [Ir(1,5-cyclooctadiene)Cl]₂ and 24.21 mg (0.021 mmol) of (S)-L4 and 5 ml of tetrahydrofuran are introduced in succession into a 10 ml Schlenk vessel. This solution is stirred at room temperature for 15 minutes. cis-3-Oxabicyclo[3.4.0]nonane-2,4-dione and 5 ml of tetrahydrofuran are introduced under an argon atmosphere into a second Schlenk vessel. The substrate solution is stirred for 5 minutes. The anhydride/Ir ratio is 100. The catalyst solution and the substrate solution are subsequently transferred in succession by means of a steel capillary into a 50 ml steel autoclave under an argon atmosphere. The autoclave is closed, the argon atmosphere is replaced by a hydrogen atmosphere in 4 flushing cycles (20 bar/1 bar). The hydrogen pressure is set to 80 bar and the autoclave is heated to 100° C. while stirring. After 15 hours, the autoclave is cooled and depressurized. The conversion and the enantiomeric purity are determined by gas chromatography. The conversion is 100% and the enantiomeric purity of the 3-oxabicyclo[3.4.0]nonan-2-one formed is 87% ee.

### EXAMPLE 7

### Preparation of 3-oxabicyclo[3.4.0]nonan-2-one

The procedure of Example 6 is repeated using 13.45 mg (0.02 mmol) of [Ir(1,5-cyclooctadiene)Cl]₂ and 35.78 mg (0.042 mmol) of (S)—(S)-L3 in place of (S)-L4. The anhydride/Ir ratio is 50, the hydrogen pressure is 80 bar and the reaction temperature is 100° C. The conversion is 99%, and the ee is 13%.

### EXAMPLE 8

### Preparation of γ-butyrolactone of the formula

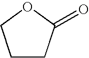

Under an argon atmosphere, 6.74 mg (0.01 mmol) [Ir(1,5-cyclooctadiene)Cl]₂ and 13.41 mg (0.021 mmol) of (R)-2,2′-diphenylphosphino-6,6′-dimethoxy-1,1′-biphenyl and 5 ml of tetrahydrofuran are introduced in succession into a 10 ml Schlenk vessel. This solution is stirred at room temperature for 15 minutes. 200.3 mg (2 mmol) of succinic anhydride and 5 ml of tetrahydrofuran are introduced under an argon atmosphere into a second Schlenk vessel. The substrate solution is stirred for 5 minutes. The anhydride/Ir ratio is 100. The catalyst solution and the substrate solution are subsequently transferred in succession by means of a steel capillary into a 50 ml steel autoclave filled with an argon atmosphere. The autoclave is closed and the argon atmosphere is replaced by a hydrogen atmosphere in 4 flushing cycles (20 bar/1 bar). The hydrogen pressure is set to 80 bar, the stirrer is switched on and the autoclave is heated to 100° C. After 15 hours, the autoclave is cooled and depressurized. The conversion is determined by gas chromatography. The conversion is 100%.

### EXAMPLE 9

### Preparation of 3-oxabicyclo[3.4.0]nonan-2-one

The procedure of Example 6 is repeated using 13.45 mg (0.02 mmol) of [Ir(1,5-cyclooctadiene)Cl]₂ and 44.23 mg (0.042 mmol) of (R)—(S)-L5 in place of (S)-L4. The anhydride/Ir ratio is 50, the hydrogen pressure is 80 bar and the reaction temperature is 100° C. The conversion is 98%, and the ee is 7%.

### EXAMPLE 10

### Preparation of 3-methyl-δ-valerolactone

The procedure of Example 2 is repeated using 22.02 mg (0.021 mmol) of (R)—(S)-L5 in place of (R)-L1. The hydrogen pressure is 70 bar, and the reaction temperature is 110° C. The conversion is 91%, and the ee is 13%.

1. Process for preparing cyclic esters, with the exception of cyclic esters of the formula 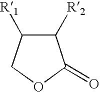
where R′₁ and R′₂ are each, independently of one another, an —NR′₃R′₄ group and R′₃ and R′₄ are each, independently of one another, hydrogen, unsubstituted or substituted alkyl, unsubstituted or substituted alkenyl, unsubstituted or substituted cycloalkyl, unsubstituted or substituted aryl, unsubstituted or aromatic-substituted aralkyl, unsubstituted or aromatic-substituted aralkenyl, cycloalkylalkyl which is unsubstituted or substituted in the cycloalkyl, heterocyclyl, unsubstituted or substituted alkanoyl, unsubstituted or substituted aroyl, unsubstituted or substituted alkylsulphonyl, unsubstituted or substituted arylsulphonyl or a silyl group Si(alkyl)₃, Si(aryl)₃ or Si(alkyl)_{1 or 2}(aryl)_{2 or 1}, or the two radicals R′₃ together are —C(O)— and the two radicals R′₄ are each, independently of one another, as defined above, by hydrogenation of a carbonyl group in at least one anhydride radical —C(O)—O—C(O)— of a cyclic dicarboxylic or polycarboxylic anhydride by means of hydrogen in the presence of a homogeneous noble metal catalyst, characterized in that the hydrogenation is carried out in a homogeneous reaction mixture using an iridium catalyst.

2. Process according to claim 1, characterized in that it is carried out at temperatures of from −20 to 150° C.

3. Process according to claim 1, characterized in that it is carried out at a pressure of from 10⁵ to 2×10⁷ Pa (pascal).

4. Process according to claim 1, characterized in that the catalyst is used in an amount of from 0.0001 to 10 mol %, based on the cyclic anhydride.

5. Process according to claim 1, characterized in that it is carried out without solvent or in the presence of an inert solvent.

6. Process according to claim 1, characterized in that the cyclic anhydride contains from one to four cyclic anhydride groups.

7. Process according to claim 6, characterized in that the cyclic anhydride group has a total of from 4 to 10 ring atoms.

8. Process according to claim 1, characterized in that the cyclic anhydride has a total of up to 60 atoms in the skeleton, with carbon atoms being able to be replaced by heteroatoms and/or groups of heteroatoms selected from the group consisting of —C(═O)—, —O—, —S—, —S(═O)—, —S(O)₂—, ═N—, —HN— and —RₐN—, where Rₐ is C₁-C₈-alkyl, C₅-C₈-cycloalkyl, C₅-C₈-cycloalkyl-C₁-C₄-alkyl, C₆-C₁₀-aryl, C₇-C₁₂-aralkyl or C₁-C₈-acyl.

9. Process according to claim 8, characterized in that the skeleton to which the at least one anhydride group —C(O)—O—C(O)— is bound comprises aliphatic, heteroaliphatic, cycloaliphatic, heterocycloaliphatic, cycloaliphatic-aliphatic, heterocycloaliphatic-aliphatic, aromatic, heteroaromatic, aromatic-aliphatic or heteroaromatic-aliphatic radicals and the cyclic radicals have from 3 to 8 ring atoms and the cyclic radicals mentioned may be bridged, fused or bridged and fused to form polycyclic radicals.

10. Process according to claim 1, characterized in that the homogeneous iridium catalysts correspond to the formulae VII and VIII,
[A₁Me₁YZ] (VII),
[A₁Me₁Y]⁺E₁⁻ (VIII),
where
A₁ represents two tertiary monophosphines or one ditertiary diphosphine which together with the Ir atom forms a five- to ten-membered ring;
Me₁ is iridium;
Y represents two olefins or one diene;
Z is Cl, Br or I; and
E₁⁻ is the anion of an oxy acid or complex acid.
